(19) United States
(12) Patent Application Publication
(10) Pub. No.: US 2023/0167463 A1
(43) Pub. Date: Jun. 1, 2023

(54) DESIGN OF TWO-PART GUIDE RNAS FOR CRISPRa APPLICATIONS

(71) Applicant: Integrated DNA Technologies, Inc., Coralville, IA (US)

(72) Inventors: Javier Alejandro Gomez Vargas, Iowa City, IA (US); Ashley Jacobi, North Liberty, IA (US); Garrett Rettig, Coralville, IA (US)

(21) Appl. No.: 17/988,275

(22) Filed: Nov. 16, 2022

Related U.S. Application Data
(60) Provisional application No. 63/279,983, filed on Nov. 16, 2021.

(51) Int. Cl. C12N 15/90; C12N 15/11; C12N 9/22

(52) U.S. Cl. CPC C12N 15/907; C12N 15/11; C12N 15/111; C12N 9/22; C12N 2310/20; C12N 2310/531; C12N 2800/80

(57) ABSTRACT

The present invention pertains to a two-component gRNA for use in a CRISPRa SAM system, wherein the two-component gRNA includes a crRNA and a tracrRNA. The crRNA and tracrRNA form a hybridized, functional gRNA in the CRISPRa SAM system. Kits and methods for using the two-component gRNA for use in a CRISPRa SAM system are also provided.

### CROSS-REFERENCE TO RELATED APPLICATIONS

This application claims benefit of priority under 35 U.S.C. 119 to U.S. Provisional Patent Application Ser. No. 63/248,300, filed Nov. 16, 2021, entitled “DESIGN OF TWO-PART GUIDE RNAS FOR CRISPRa APPLICATIONS,” the contents of which is herein incorporated by reference in its entirety.

### SEQUENCE LISTING

The instant application contains a Sequence Listing which has been submitted in XML format via Patent Center and is hereby incorporated by reference in its entirety. Said XML copy, created on Jan. 19, 2023, is named IDT01-022-US.xml, and is 105,999 bytes in size.

### FIELD OF THE INVENTION

This invention pertains to methods and compositions for the design of two-part guide RNA's for CRISPRa Synergistic Activation Mediator applications.

### BACKGROUND OF THE INVENTION

The clustered, regularly interspaced short palindromic repeat (CRISPR) gene editing systems are important biotechnology tools having a wide range of applications. The CRISPR-Cas9 system is currently being used in molecular genetics to precisely make changes to genomic DNA. In addition to the conventional, targeted nuclease activity, this technology can also be used to activate or interfere with transcriptional activity of a gene. CRISPR activation (CRISPRa) is a genetic tool that allows the CRISPR-Cas9 system, using guide RNAs, to transcriptionally activate a specific gene. CRISPRa Synergistic Activation Mediator (SAM) is a modality of CRISPRa where a catalytically dead Cas9 (dCas9) fused to VP64 uses an sgRNA with a minimal hairpin MS2-binding aptamer which binds dimerized MS2 bacteriophage coat proteins fused to the activation domains of NF-κB trans-activating subunit p65 (p65) and human heat-shock factor 1 (HSF1). This binding cascade of transcriptional activators results in upregulation of gene expression guided by a single guide RNA (sgRNA) which typically are about 150-nt in length.

CRISPRa SAM displays advantages over other CRISPRa systems. Other CRISPRa systems require pooling of guides to significantly upregulate a target. This decreases the scalability of the system and its use in large genetic screens. CRISPRa SAM uses a single sgRNA. Konnerman, 2015, provides many sgRNA variations. These variations display differences in the upregulation of their target genes.

Given the complexity and difficulty in synthesizing long sgRNAs for such CRISPRa systems, it would be desirable to design a guide RNA (gRNA) as two RNA molecules, as with the classic CRISPR-Cas9 system.

### BRIEF SUMMARY OF THE INVENTION

In a first aspect, a two-component gRNA for use in a CRISPRa SAM system is provided. The two-component gRNA includes a crRNA and a tracrRNA. The crRNA and tracrRNA form a hybridized, functional gRNA in the CRISPRa SAM system.

In a second aspect, a kit for use in a CRISPRa SAM system is provided. The kit includes a two-component gRNA for use in a CRISPRa SAM system. The two-component gRNA includes a crRNA and a tracrRNA. The crRNA and tracrRNA form a hybridized, functional gRNA in the CRISPRa SAM system.

In a third aspect, a method of using a CRISPRa SAM system in a cell is provided. A first step includes introducing a two-component gRNA of a CRISPRa SAM system into the cell. The two-component gRNA includes a crRNA; and a tracrRNA. The crRNA and tracrRNA form a hybridized, functional gRNA in the CRISPRa SAM system.

### BRIEF DESCRIPTION OF THE DRAWINGS

FIG. 1A depicts the sgRNA sequence, specifically sgRNA 2.0. with the important folds, sequence and structure adapted from Konnerman, 2015.

FIG. 1B depicts the folded sgRNA targeting the Fatty Acid Synthase gene (FAS), wherein the designated marks correspond to the bases where the guide RNA was split into two parts.

FIG. 1C depicts the folding structure of the proposed crRNA, denoted as crRNA (2.0) pt 1.1.

FIG. 1D depicts the folding structure of the proposed tracrRNA, denoted as tracrRNA(2.0) 1.2.

FIG. 1E depicts the folding structure of the proposed crRNA, denoted as crRNA (2.0) pt2.1.

FIG. 1F depicts the folding structure of the proposed tracrRNA, denoted as tracrRNA(2.0) pt 2.2.

FIG. 1G depicts a folded modified version of the sgRNA targeting the Fatty Acid Synthase gene (FAS), wherein the designated marks correspond to the bases where the guide RNA was split into two parts.

FIG. 111 depicts the folding structure of the proposed crRNA, denoted as crRNA (D5) pt 1.1.

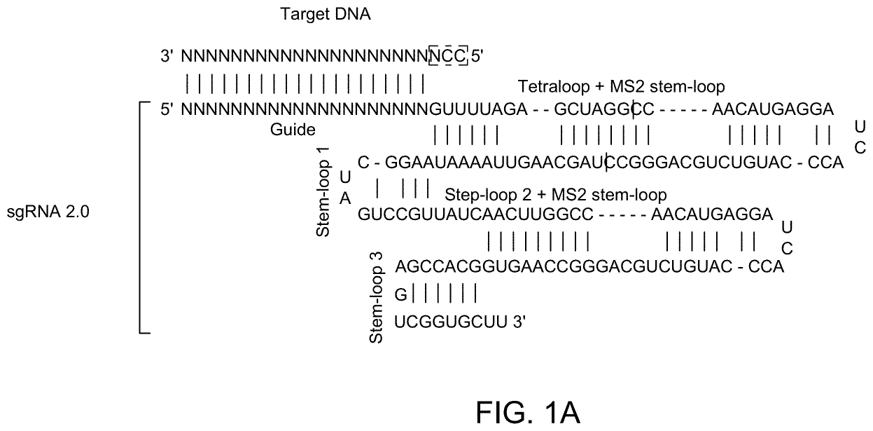

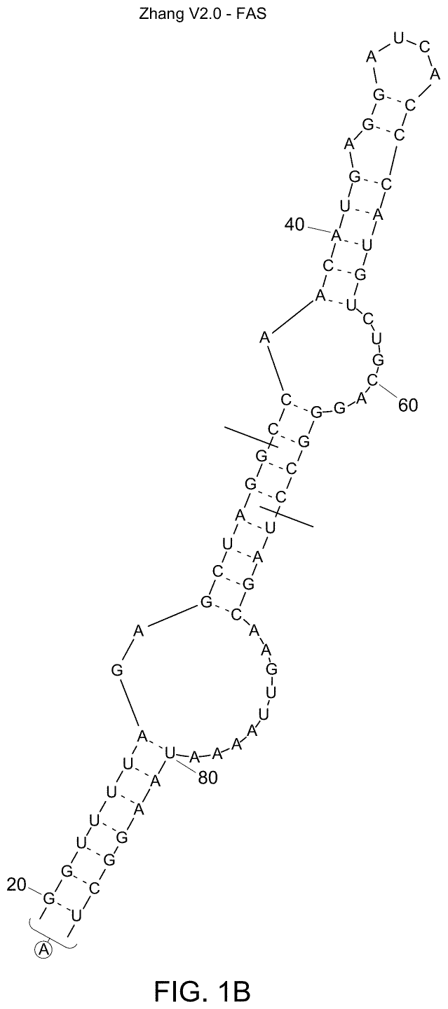

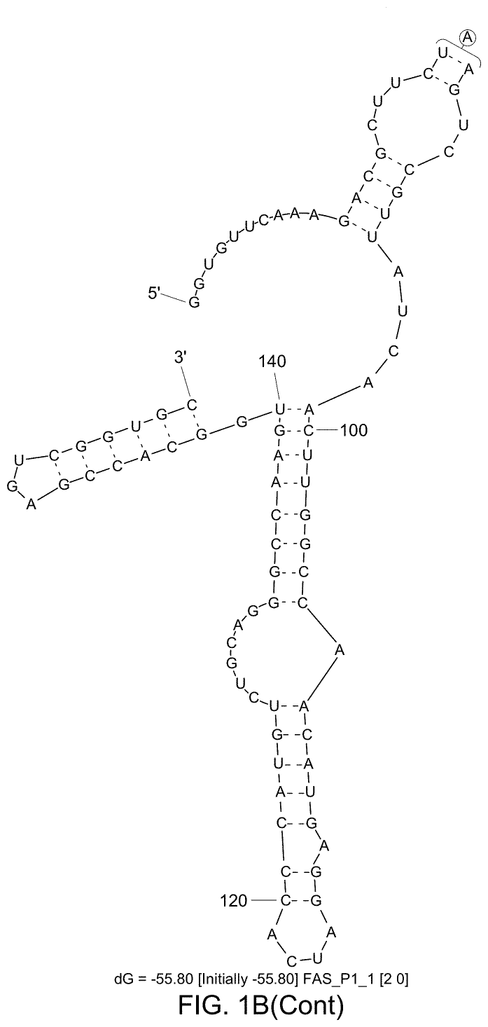

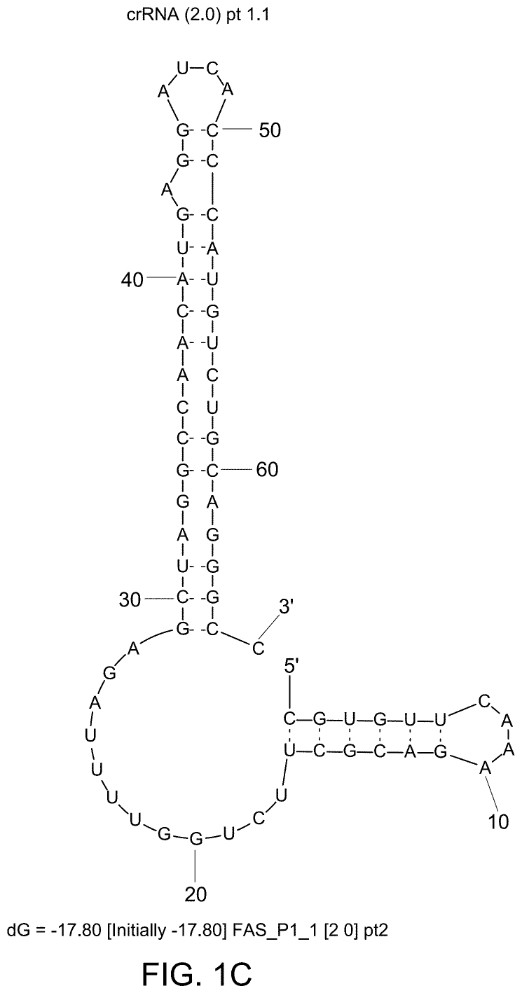

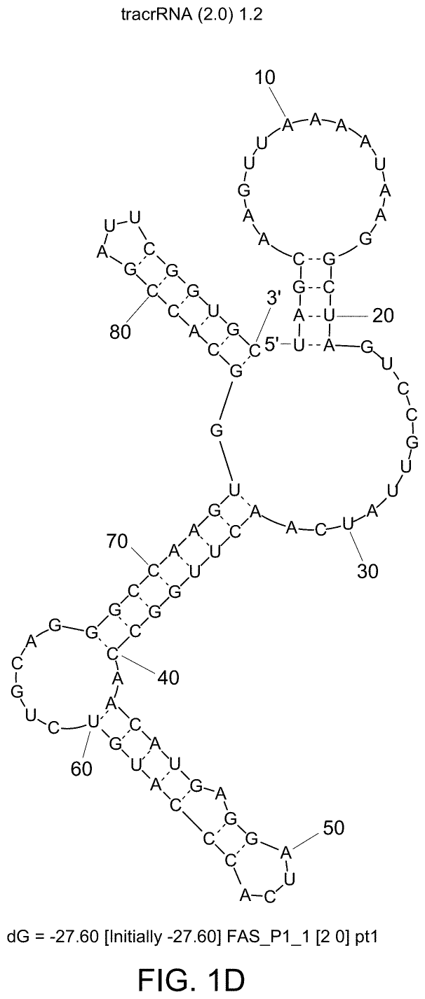

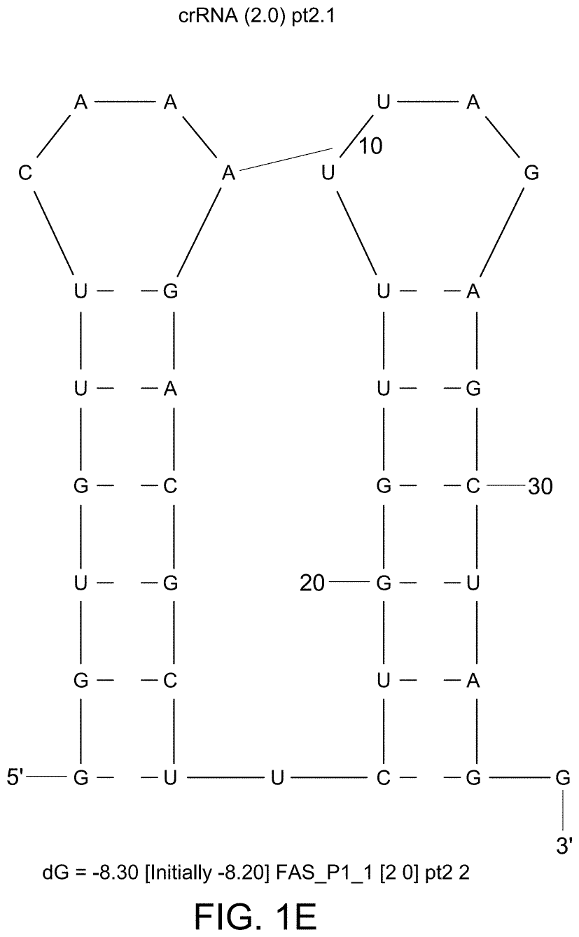

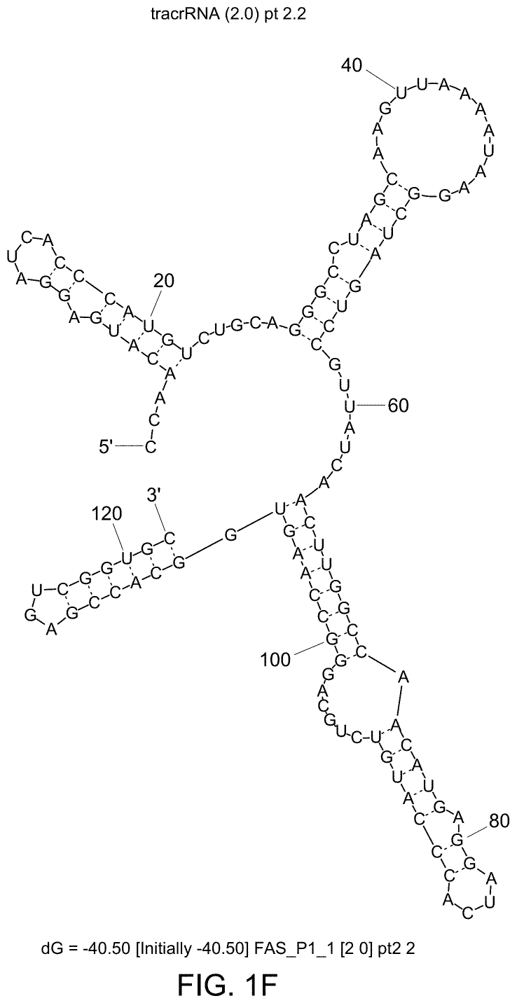

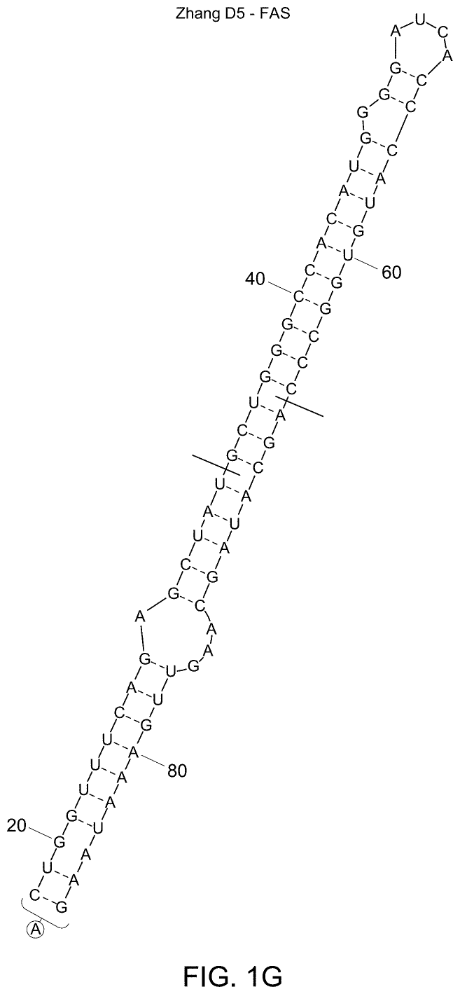

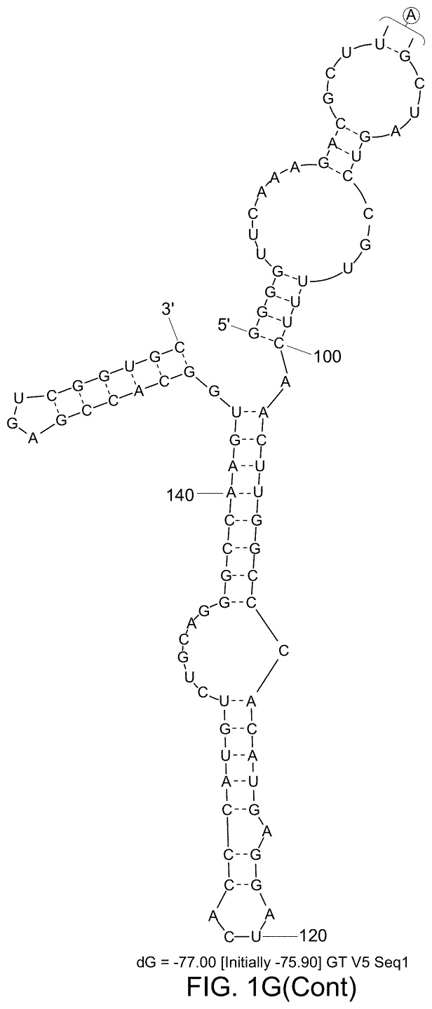

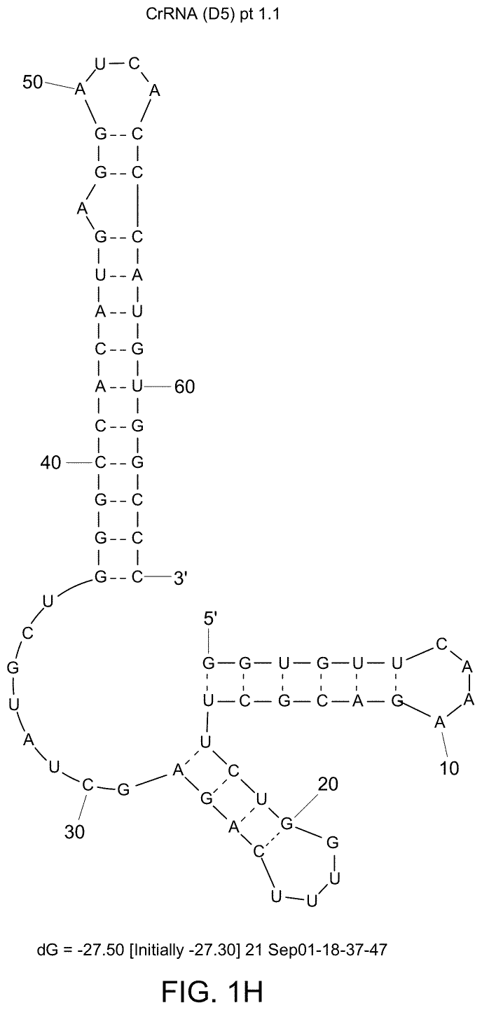

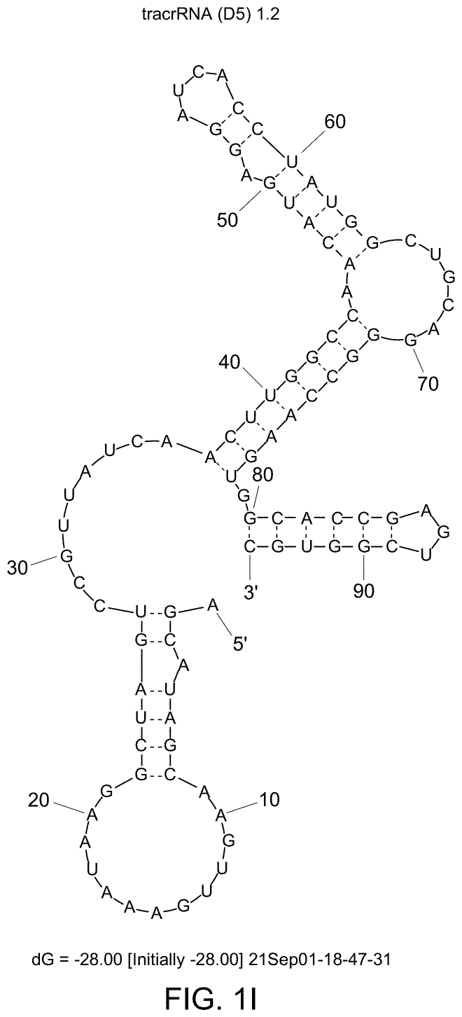

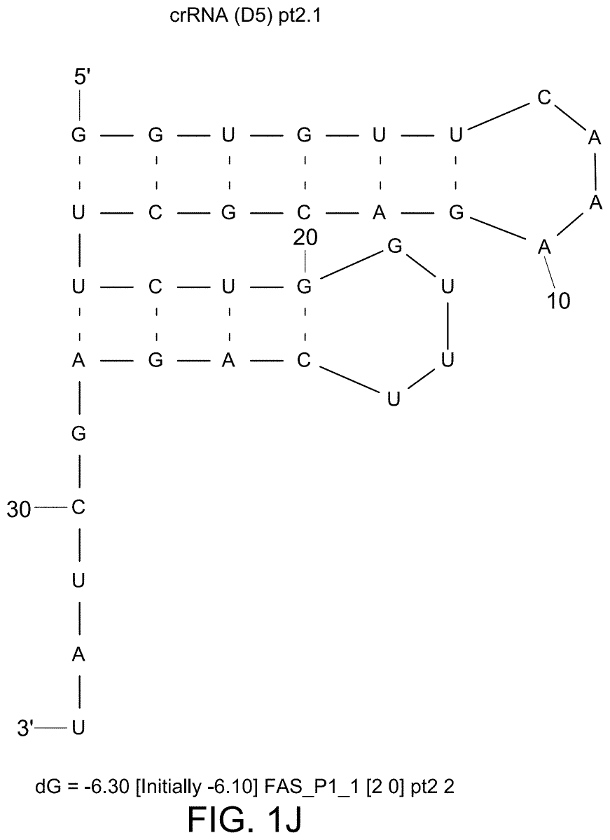

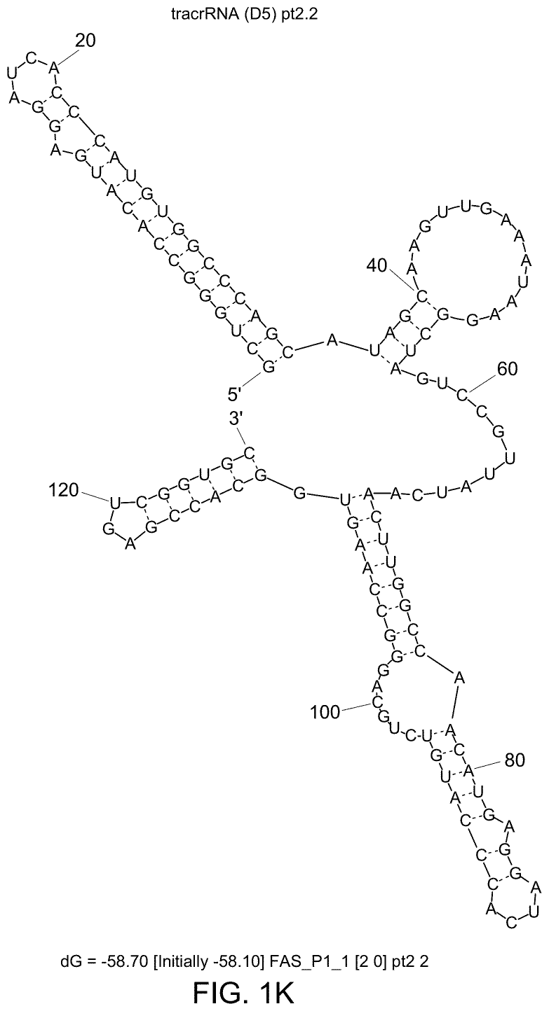

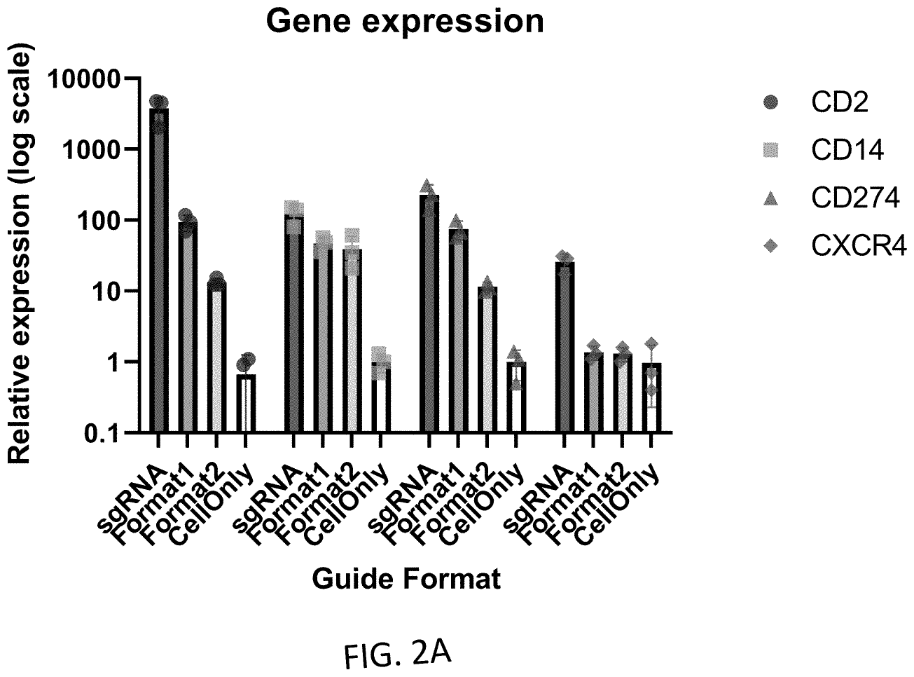

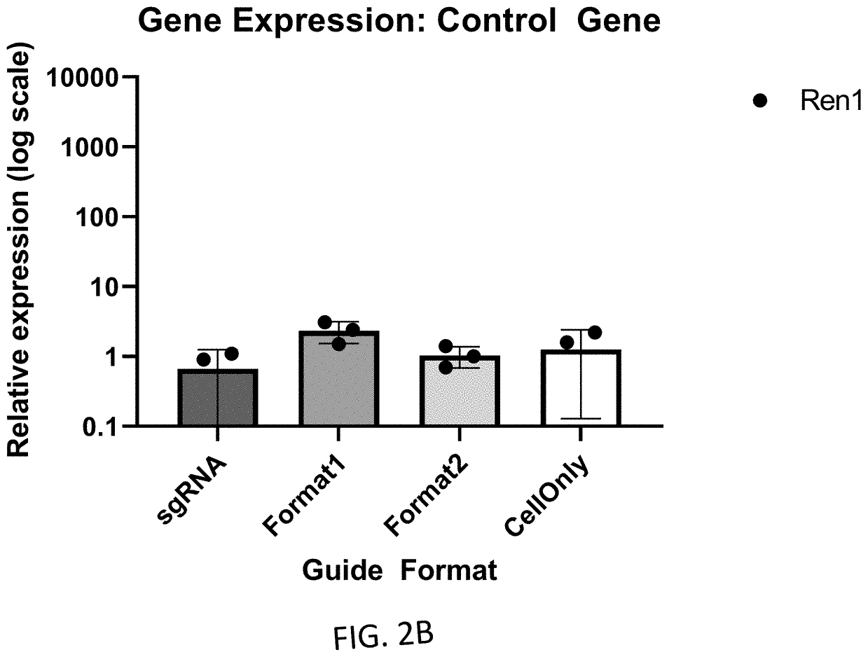

FIG. 1I depicts the folding structure of the proposed tracrRNA, denoted as tracrRNA (D5) 1.2.

FIG. 1J depicts the folding structure of the proposed crRNA, denoted as crRNA (D5) 02.1.

FIG. 1K depicts the folding structure of the proposed tracrRNA, denoted as tracrRNA (D5) pt 2.1.

FIG. 2A depicts exemplary data showing sgRNA format can provide increased gene expression. Each gene corresponds to a different colored shape (CD2, blue circles; CD14, green square; CD274, red triangle; CXCR4, purple diamonds) and each guide format corresponds to a different shades of gray (darkest grey for sgRNA; lighter grey format 1; lightest grey for format 2; white for cells only). The 2-component guide formats induce less expression than sgRNA. However, Format 1 induces higher expression compared to Format 2.

FIG. 2B depicts the corresponding data for Ren1 gene expression, which is a negative control, that does not display any increase in gene expression, regardless of the guide format.

### DETAILED DESCRIPTION OF THE INVENTION

The current invention provides novel designs of two-component guide RNA (gRNA) for CRISPRa SAM systems, wherein the target specific portion (crRNA) and the universal scaffold (tracrRNA) are synthesized as separate RNA components and thereafter hybridized together to reconstitute a functional gRNA derived from a sgRNA for use in CRISPRa SAM systems. This provides flexibility in testing multiple gene targets, by synthesizing a much smaller crRNA and using a common tracrRNA. Splitting an sgRNA, therefore, provides an attractive strategy for high throughput testing of multiple guide RNAs using CRISPRa SAM system. We set out to provide a design strategy to split a traditional sgRNA in the CRISPRa SAM system, into two parts. We used RNA folding software, UNAfold to determine where to split the CRISPRa sgRNA without compromising the important secondary structures and features, like the stem-loop recruitment domains for MS2 (see FIG. 1), as well as critical secondary structures inherent to the tracrRNA's affinity for Cas9.

In one embodiment, the guide RNA can be a two part system comprising a crRNA and a tracrRNA. In one respect, the tetraloop and loop 2 of the tracrRNA are modified by the insertion of distinct aptamer sequences. In another aspect the distinct aptamer sequence is an RNA aptamer sequence which is added to the sgRNA tetraloop and stem-loop sequences.

In another respect, the distinct aptamer sequence binds to one or more adaptor proteins. In another aspect the RNA aptamer selectively binds dimerized MS2 bacteriophage coat proteins.

In another embodiment, the two part guide RNA system may comprise different length crRNA and tracrRNA. In one aspect the two part guide RNA system is designed such that the secondary structures and features maintain function. In one aspect the crRNA is 32 bp to 68 bp in length. In another aspect the crRNA is optimally 34 bp or 66 bp in length. In another aspect the tracrRNA is 88 bp to 124 bp in length. In another aspect the tracrRNA is optimally 90 bp or 122 bp in length.

In another embodiment, the sgRNA are split at locations to preserve the function of the MS2 stem loop sequence and the tetraloop and loop 2 of the tracrRNA.

In order to protect and increase stability, the gRNAs were chemically modified. This follows previous strategies where one or more phosphorothioate linkages and 2′-O-methyl RNA bases are incorporated in the 3 terminal nucleotides at the 5′ and 3′ ends of both the crRNAs and the tracrRNAs. We designed four tracrRNA sequences and twenty-eight crRNA sequences in order to test the 2 RNA component designs and verify the efficacy of our designs in a biological assay (see SEQ ID Nos. of Table 1).

TABLE 1
Proof of concept sequences to test CRISPRa two-part system.

| SEQ | | |
|---|---|---|
| ID | | |
| NO: | Sequence Name | RNA Sequence¹ |
| 1 | (2.0) pt1.2 tracrRNA | mU*mA*mG*rCrArArGrUrUrArArArArUrArArGrGrCrUrArGrUrCrC |
| | | rGrUrUrArUrCrArArCrUrUrGrGrCrCrArArCrArUrGrArGrGrArUrCr |
| | | ArCrCrCrArUrGrUrCrUrGrCrArGrGrGrCrCrArArGrUrGrGrCrArCr |
| | | CrGrArGrUrCrGrG*mU*mG*mC |
| 2 | FAS_P1_1 (2.0) pt1.1 crRNA | mG*mG*mU*rGrUrUrCrArArArGrArCrGrCrUrUrCrUrGrGrUrUrUr |
| | | UrArGrArGrCrUrArGrGrCrCrArArCrArUrGrArGrGrArUrCrArCrCr |
| | | CrArUrGrUrCrUrGrCrArGrG*mG*mC*mC |
| 3 | CD274_19h6 (2.0) pt1.1 | mG*mU*mC*rArGrGrArArArGrUrCrCrArArCrGrCrCrGrUrUrUrUr |
| | crRNA | ArGrArGrCrUrArGrGrCrCrArArCrArUrGrArGrGrArUrCrArCrCrCr |
| | | ArUrGrUrCrUrGrCrArGrG*mG*mC*mC |
| 4 | CD14_h1 (2.0) pt1.1 crRNA | mU*mA*mA*rCrArGrGrArArGrGrArUrUrCrUrGrCrArGrUrUrUrUrA |
| | | rGrArGrCrUrArGrGrCrCrArArCrArUrGrArGrGrArUrCrArCrCrCrAr |
| | | UrGrUrCrUrGrCrArGrG*mG*mC*mC |
| 5 | CD2-sg3 (2.0) pt1.1 crRNA | mA*mC*mU*rGrUrArArArArGrArUrGrUrArArArGrArGrGrUrUrUrU |
| | | rArGrArGrCrUrArGrGrCrCrArArCrArUrGrArGrGrArUrCrArCrCrCr |
| | | ArUrGrUrCrUrGrCrArGrG*mG*mC*mC |
| 6 | CXCR4_14h6 (2.0) pt1.1 | mG*mG*mG*rArGrGrUrCrCrUrGrUrCrCrGrGrCrUrCrGrUrUrUrUr |
| | crRNA | ArGrArGrCrUrArGrGrCrCrArArCrArUrGrArGrGrArUrCrArCrCrCr |
| | | ArUrGrUrCrUrGrCrArGrG*mG*mC*mC |
| 7 | Ren1 (2.0) pt1.1 crRNA | mC*mG*mA*rCrCrCrCrGrArGrCrArGrCrGrGrArArGrGrUrUrUrUr |
| | | ArGrArGrCrUrArGrGrCrCrArArCrArUrGrArGrGrArUrCrArCrCrCr |
| | | ArUrGrUrCrUrGrCrArGrG*mG*mC*mC |
| 8 | gREP2 (2.0) pt1.1 crRNA | mC*mG*mU*rCrGrGrGrArUrCrArArCrUrCrGrArArCrGrUrUrUrUr |
| | | ArGrArGrCrUrArGrGrCrCrArArCrArUrGrArGrGrArUrCrArCrCrCr |
| | | ArUrGrUrCrUrGrCrArGrG*mG*mC*mC |
| 9 | (2.0) pt2.2 tracrRNA | mC*mC*mA*rArCrArUrGrArGrGrArUrCrArCrCrCrArUrGrUrCrUrG |
| | | rCrArGrGrGrCrCrUrArGrCrArArGrUrUrArArArArUrArArGrGrCrUr |
| | | ArGrUrCrCrGrUrUrArUrCrArArCrUrUrGrGrCrCrArArCrArUrGrAr |
| | | GrGrArUrCrArCrCrCrArUrGrUrCrUrGrCrArGrGrGrCrCrArArGrUr |
| | | GrGrCrArCrCrGrArGrUrCrGrG*mU*mG*mC |
| 10 | FAS_P1_1 (2.0) pt2.1 crRNA | mG*mG*mU*rGrUrUrCrArArArGrArCrGrCrUrUrCrUrGrGrUrUrUr |
| | | UrArGrArGrCrU*mA*mG*mG |
| 11 | CD274_19h6 (2.0) pt2.1 | mG*mU*mC*rArGrGrArArArGrUrCrCrArArCrGrCrCrGrUrUrUrUr |
| | crRNA | ArGrArGrCrU*mA*mG*mG |
| 12 | CD14_h1 (2.0) pt2.1 crRNA | mU*mA*mA*rCrArGrGrArArGrGrArUrUrCrUrGrCrArGrUrUrUrUrA |
| | | rGrArGrCrU*mA*mG*mG |
| 13 | CD2-sg3 (2.0) pt2.1 crRNA | mA*mC*mU*rGrUrArArArArGrArUrGrUrArArArGrArGrGrUrUrUrU |
| | | rArGrArGrCrU*mA*mG*mG |
| 14 | CXCR4_14h6 (2.0) pt2.1 | mG*mG*mG*rArGrGrUrCrCrUrGrUrCrCrGrGrCrUrCrGrUrUrUrUr |
| | crRNA | ArGrArGrCrU*mA*mG*mG |
| 15 | Ren1 (2.0) pt2.1 crRNA | mC*mG*mA*rCrCrCrCrGrArGrCrArGrCrGrGrArArGrGrUrUrUrUr |
| | | ArGrArGrCrU*mA*mG*mG |
| 16 | gREP2 (2.0) pt2.1 crRNA | mC*mG*mU*rCrGrGrGrArUrCrArArCrUrCrGrArArCrGrUrUrUrUr |
| | | ArGrArGrCrU*mA*mG*mG |
| 17 | (D5) pt1.2 tracrRNA | mA*mG*mC*rArUrArGrCrArArGrUrUrGrArArArUrArArGrGrCrUrA |
| | | rGrUrCrCrGrUrUrArUrCrArArCrUrUrGrGrCrCrArArCrArUrGrArGr |
| | | GrArUrCrArCrCrCrArUrGrUrCrUrGrCrArGrGrGrCrCrArArGrUrGr |
| | | GrCrArCrCrGrArGrUrCrGrG*mU*mG*mC |
| 18 | FAS_P1_1 (D5) pt1.1 crRNA | mG*mG*mU*rGrUrUrCrArArArGrArCrGrCrUrUrCrUrGrGrUrUrUr |
| | | CrArGrArGrCrUrArUrGrCrUrGrGrGrCrCrArCrArUrGrArGrGrArUr |
| | | CrArCrCrCrArUrGrUrGrG*mC*mC*mC |
| 19 | CD274_19h6 (D5) pt1.1 | mG*mU*mC*rArGrGrArArArGrUrCrCrArArCrGrCrCrGrUrUrUrCr |
| | crRNA | ArGrArGrCrUrArUrGrCrUrGrGrGrCrCrArCrArUrGrArGrGrArUrCr |
| | | ArCrCrCrArUrGrUrGrG*mC*mC*mC |
| 20 | CD14_h1 (D5) pt1.1 crRNA | mU*mA*mA*rCrArGrGrArArGrGrArUrUrCrUrGrCrArGrUrUrUrCrA |
| | | rGrArGrCrUrArUrGrCrUrGrGrGrCrCrArCrArUrGrArGrGrArUrCrAr |
| | | CrCrCrArUrGrUrGrG*mC*mC*mC |
| 21 | CD2-sg3 (D5) pt1.1 crRNA | mA*mC*mU*rGrUrArArArArGrArUrGrUrArArArGrArGrGrUrUrUrC |
| | | rArGrArGrCrUrArUrGrCrUrGrGrGrCrCrArCrArUrGrArGrGrArUrCr |
| | | ArCrCrCrArUrGrUrGrG*mC*mC*mC |
| 22 | CXCR4_14h6 (D5) pt1.1 | mG*mG*mG*rArGrGrUrCrCrUrGrUrCrCrGrGrCrUrCrGrUrUrUrCr |
| | crRNA | ArGrArGrCrUrArUrGrCrUrGrGrGrCrCrArCrArUrGrArGrGrArUrCr |
| | | ArCrCrCrArUrGrUrGrG*mC*mC*mC |
| 23 | Ren1 (D5) pt1.1 crRNA | mC*mG*mA*rCrCrCrCrGrArGrCrArGrCrGrGrArArGrGrUrUrUrCr |
| | | ArGrArGrCrUrArUrGrCrUrGrGrGrCrCrArCrArUrGrArGrGrArUrCr |
| | | ArCrCrCrArUrGrUrGrG*mC*mC*mC |
| 24 | gREP2 (D5) pt1.1 crRNA | mC*mG*mU*rCrGrGrGrArUrCrArArCrUrCrGrArArCrGrUrUrUrCr |
| | | ArGrArGrCrUrArUrGrCrUrGrGrGrCrCrArCrArUrGrArGrGrArUrCr |
| | | ArCrCrCrArUrGrUrGrG*mC*mC*mC |
| 25 | (D5) pt2.2 tracrRNA | mG*mC*mU*rGrGrGrCrCrArCrArUrGrArGrGrArUrCrArCrCrCrAr |
| | | UrGrUrGrGrCrCrCrArGrCrArUrArGrCrArArGrUrUrGrArArArUrAr |
| | | ArGrGrCrUrArGrUrCrCrGrUrUrArUrCrArArCrUrUrGrGrCrCrArAr |
| | | CrArUrGrArGrGrArUrCrArCrCrCrArUrGrUrCrUrGrCrArGrGrGrCr |
| | | CrArArGrUrGrGrCrArCrCrGrArGrUrCrGrG*mU*mG*mC |
| 26 | FAS_P1_1 (D5) pt2.1 crRNA | mG*mG*mU*rGrUrUrCrArArArGrArCrGrCrUrUrCrUrGrGrUrUrUr |
| | | CrArGrArGrC*mU*mA*mU |
| 27 | CD274_19h6 (D5) pt2.1 | mG*mU*mC*rArGrGrArArArGrUrCrCrArArCrGrCrCrGrUrUrUrCr |
| | crRNA | ArGrArGrC*mU*mA*mU |
| 28 | CD14_h1 (D5) pt2.1 crRNA | mU*mA*mA*rCrArGrGrArArGrGrArUrUrCrUrGrCrArGrUrUrUrCrA |
| | | rGrArC*mU*mA*mU |
| 29 | CD2-sg3 (D5) pt2.1 crRNA | mA*mC*mU*rGrUrArArArArGrArUrGrUrArArArGrArGrGrUrUrUrC |
| | | rArGrArGrC*mU*mA*mU |
| 30 | CXCR4_14h6 (D5) pt2.1 | mG*mG*mG*rArGrGrUrCrCrUrGrUrCrCrGrGrCrUrCrGrUrUrUrCr |
| | crRNA | ArGrArGrC*mU*mA*mU |
| 31 | Ren1 (D5) pt2.1 crRNA | mC*mG*mA*rCrCrCrCrGrArGrCrArGrCrGrGrArArGrGrUrUrUrCr |
| | | ArGrArGrC*mU*mA*mU |
| 32 | gREP2 (D5) pt2.1 crRNA | mC*mG*mU*rCrGrGrGrArUrCrArArCrUrCrGrArArCrGrUrUrUrCr |
| | | ArGrArGrC*mU*mA*mU |

¹Key: *, phosphorothioate; m, 2’-O-methyl moiety; rA, rG, rU, and rC, ribonucleosides.

### EXAMPLES

### Example 1. Evaluation of Guide RNA Formats

To test guide formats, we performed an experiment using a modified HEK293 cell line (BPSBioscience, #78192). These cells stably express a deadCas9 construct fused to VP64 under blasticidin resistance. They also stably express the P65-HSF1-MS2 construct under Hygromycin resistance. We nucleofected these cells using a Lonza 4D Nucleofector to determine which of our two guide formats, Format 1 (1.1, 1.2) or Format 2 (2.1,2.2), was most effective at upregulating gene expression.

The two-part guides were annealed to their respective tracrRNA in equimolar amounts, heated, and annealed. Cells were nucleofected with guides targeting Ren1 (negative control), CD2, CD14, CD274 and CXCR4 in either single guide RNA format or Format 1 or 2 of our two-part system. Additionally, a Cells Only control was included. Guides were delivered at a standard dose of 1 μM per guide and cells were plated post-transfection in triplicate for 48 hours before total RNA was collected. cDNA synthesis proceeded using standard IDT protocols with a SuperScript II reverse transcriptase.

Quantitative qPCR was performed using IDT's 2× PrimeTime master mix and TaqMan assays. Each biological replicate was run in triplicate and for each sample housekeeping genes HPRT and SFRS9 were used to normalize gene expression.

For each sample, the average of the technical replicate was obtained, then normalized to the average of the corresponding housekeeping genes. The Cells Only control was used as a baseline for expression of each gene and the data is reported as expression relative to the Cells Only control.

Referring to FIG. 2, s comparison of each guide format is provided. Each gene corresponds to a different colored shape (CD2, blue circles; CD14, green square; CD274, red triangle; CXCR4, purple diamonds) and each guide format corresponds to a different shades of gray (darkest grey for sgRNA; lighter grey format 1; lightest grey for format 2; white for cells only). The sgRNA format shows a strong increase in gene expression (FIG. 2A). The 2-guide formats induce less expression than sgRNA. However, Format 1 induces higher expression compared to Format 2. Ren1 gene expression, which is a negative control, does not show any increase in gene expression, regardless of the guide format (FIG. 2B).

### Applications

In a first aspect, a two-component gRNA for use in a CRISPRa SAM system is provided. The two-component gRNA includes a crRNA and a tracrRNA. The crRNA and tracrRNA form a hybridized, functional gRNA in the CRISPRa SAM system. In a first respect, the tracrRNA is modified with the insertion of an aptamer sequence. As a further refinement of the first respect, the aptamer sequence is inserted between the tetraloop sequence and the loop 2 sequence. As a further refinement of the first respect, the aptamer sequence is inserted between the loop 2 sequence and the loop 3 sequence. As a further refinement of the first respect, the aptamer sequence is a MS2 stem loop. In a second respect, the tracrRNA is selected from SEQ ID NO: 1, 9, 17, and 25.

In a second aspect, a kit for use in a CRISPRa SAM system is provided. The kit includes a two-component gRNA for use in a CRISPRa SAM system. The two-component gRNA includes a crRNA and a tracrRNA. The crRNA and tracrRNA form a hybridized, functional gRNA in the CRISPRa SAM system. In a first respect, the tracrRNA is modified with the insertion of an aptamer sequence. In a second respect, the aptamer sequence is inserted between the tetraloop sequence and the loop 2 sequence or wherein the aptamer sequence is inserted between the loop 2 sequence and the loop 3 sequence. In a third respect, the aptamer sequence is a MS2 stem loop. In a fourth respect, the tracrRNA is selected from SEQ ID NO: 1, 9, 17, and 25.

In a third aspect, a method of using a CRISPRa SAM system in a cell is provided. A first step includes introducing a two-component gRNA of a CRISPRa SAM system into the cell. The two-component gRNA includes a crRNA; and a tracrRNA. The crRNA and tracrRNA form a hybridized, functional gRNA in the CRISPRa SAM system. In a first respect, the tracrRNA is modified with the insertion of an aptamer sequence. In a second respect, the aptamer sequence is inserted between the tetraloop sequence and the loop 2 sequence or wherein the aptamer sequence is inserted between the loop 2 sequence and the loop 3 sequence. In a third respect, the aptamer sequence is a MS2 stem loop. In a fourth respect, the tracrRNA is selected from SEQ ID NO: 1, 9, 17, and 25.

### Definitions

To aid in understanding the invention, several terms are defined below.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The terms “nucleic acid” and “oligonucleotide,” as used herein, refer to polydeoxyribonucleotides (containing 2-deoxy-D-ribose), polyribonucleotides (containing D-ribose), and to any other type of polynucleotide which is an N glycoside of a purine or pyrimidine base. There is no intended distinction in length between the terms “nucleic acid,” “oligonucleotide,” and “polynucleotide,” and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The terms “target, “target sequence,” “target region,” and “target nucleic acid,” as used herein, are synonymous and refer to a region or sequence of a nucleic acid which is to be amplified, sequenced or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions.” Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, N.Y.; Wetmur, 1991, Critical Review in Biochem. and Mol. Biol. 26(3/4):227-259; and Owczarzy et al., 2008, Biochemistry, 47: 5336-5353, which are incorporated herein by reference).

### REFERENCES CITED

Konermann S, Brigham M D, Trevino A E, Joung J, Abudayyeh O O, Barcena C, Hsu P D, Habib N, Gootenberg J S, Nishimasu H, Nureki O, Zhang F. Genome-scale transcriptional activation by an engineered CRISPR-Cas9 complex. Nature. 2015 Jan. 29; 517(7536):583-8. doi: 10.1038/nature14136. Epub 2014 Dec. 10. PMID: 25494202; PMCID: PMC4420636.
Strezoska Ž, Dickerson S M, Maksimova E, Chou E, Gross M M, Hemphill K, Hardcastle T, Perkett M, Stombaugh J, Miller G W, Anderson E M, Vermeulen A, Smith A V B. CRISPR-mediated transcriptional activation with synthetic guide RNA. J Biotechnol. 2020 Aug. 10; 319:25-35. doi: 10.1016/j.jbiotec.2020.05.005. Epub 2020 May 27. PMID: 32470463.
Chylinski K, Le Rhun A, Charpentier E. The tracrRNA and Cas9 families of type II CRISPR-Cas immunity systems. RNA Biol. 2013 May; 10(5):726-37. doi: 10.4161/rna.24321. Epub 2013 Apr. 5. PMID: 23563642; PMCID: PMC3737331.
Lim Y, Bak S Y, Sung K, Jeong E, Lee S H, Kim J S, Bae S, Kim S K. Structural roles of guide RNAs in the nuclease activity of Cas9 endonuclease. Nat Commun. 2016 Nov. 2; 7:13350. doi: 10.1038/ncomms13350. PMID: 27804953; PMCID: PMC5097132.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A two-component gRNA for use in a CRISPRa SAM system, wherein the two-component gRNA comprises:
a crRNA; and
a tracrRNA,
wherein the crRNA and tracrRNA form a hybridized, functional gRNA in the CRISPRa SAM system.

2. The two component gRNA of claim 1, wherein the tracrRNA is modified with the insertion of an aptamer sequence.

3. The tracrRNA of claim 2, wherein the aptamer sequence is inserted between the tetraloop sequence and the loop 2 sequence or wherein the aptamer sequence is inserted between the loop 2 sequence and the loop 3 sequence.

4. The aptamer sequence of claim 3, wherein the aptamer sequence is a MS2 stem loop.

5. The two-component gRNA of claim 1, wherein the tracrRNA is selected from SEQ ID NO: 1, 9, 17, and 25.

6. A kit for use in a CRISPRa SAM system, comprising:
a two-component gRNA for use in a CRISPRa SAM system, wherein the two-component gRNA comprises:
a crRNA; and
a tracrRNA,
wherein the crRNA and tracrRNA form a hybridized, functional gRNA in the CRISPRa SAM system.

7. The two component gRNA of claim 6, wherein the tracrRNA is modified with the insertion of an aptamer sequence.

8. The tracrRNA of claim 7, wherein the aptamer sequence is inserted between the tetraloop sequence and the loop 2 sequence or wherein the aptamer sequence is inserted between the loop 2 sequence and the loop 3 sequence.

9. The aptamer sequence of claim 8, wherein the aptamer sequence is a MS2 stem loop.

10. The two-component gRNA of claim 6, wherein the tracrRNA is selected from SEQ ID NO: 1, 9, 17, and 25.

11. A method of using a CRISPRa SAM system in a cell, comprising:
introducing a two-component gRNA of a CRISPRa SAM system into the cell, wherein the two-component gRNA comprises:
a crRNA; and
a tracrRNA,
wherein the crRNA and tracrRNA form a hybridized, functional gRNA in the CRISPRa SAM system.

12. The method of claim 11, wherein the tracrRNA is modified with the insertion of an aptamer sequence.

13. The method of claim 12, wherein the aptamer sequence is inserted between the tetraloop sequence and the loop 2 sequence or wherein the aptamer sequence is inserted between the loop 2 sequence and the loop 3 sequence.

14. The method of claim 13, wherein the aptamer sequence is a MS2 stem loop.

15. The method of claim 11, wherein the tracrRNA is selected from SEQ ID NO: 1, 9, 17, and 25.


SEQUENCE LISTING (32 sequences)

SEQ ID NO: 1 - RNA, 90 nt, synthetic construct
  source 1..90: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 88: note = 2-O-methyl ribose moiety and phosphorothioate
  misc_feature 89: note = 2-O-methyl ribose moiety and phosphorothioate
  misc_feature 90: note = 2-O-methyl ribose moiety and phosphorothioate
tagcaagttaaaataaggctagtccgttatcaacttggccaacatgaggatcacccatgtctgcagggccaagtggcaccgagtcggtgc

SEQ ID NO: 2 - RNA, 65 nt, synthetic construct
  source 1..65: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
  misc_feature 65: note = 2-O-methyl ribose moiety and phosphorothioate
ggtgttcaaagacgcttctggttttagagctaggccaacatgaggatcacccatgtctgcaggcc

SEQ ID NO: 3 - RNA, 65 nt, synthetic construct
  source 1..65: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
  misc_feature 65: note = 2-O-methyl ribose moiety and phosphorothioate
gtcaggaaagtccaacgccgttttagagctaggccaacatgaggatcacccatgtctgcagggcc

SEQ ID NO: 4 - RNA, 65 nt, synthetic construct
  source 1..65: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
  misc_feature 65: note = 2-O-methyl ribose moiety and phosphorothioate
taacaggaaggattctgcagttttagagctaggccaacatgaggatcacccatgtctgcagggcc

SEQ ID NO: 5 - RNA, 66 nt, synthetic construct
  source 1..66: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 64: note = 2-O-methyl ribose moiety and phosphorothioate
  misc_feature 65: note = 2-O-methyl ribose moiety and phosphorothioate
  misc_feature 66: note = 2-O-methyl ribose moiety and phosphorothioate
actgtaaaagatgtaaagaggttttagagctaggccaacatgaggatcacccatgtctgcagggcc

SEQ ID NO: 6 - RNA, 65 nt, synthetic construct
  source 1..65: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
  misc_feature 65: note = 2-O-methyl ribose moiety and phosphorothioate
gggaggtcctgtccggctcgttttagagctaggccaacatgaggatcacccatgtctgcagggcc

SEQ ID NO: 7 - RNA, 65 nt, synthetic construct
  source 1..65: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
  misc_feature 65: note = 2-O-methyl ribose moiety and phosphorothioate
cgaccccgagcagcggaaggttttagagctaggccaacatgaggatcacccatgtctgcagggcc

SEQ ID NO: 8 - RNA, 65 nt, synthetic construct
  source 1..65: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
  misc_feature 65: note = 2-O-methyl ribose moiety and phosphorothioate
cgtcgggatcaactcgaacgttttagagctaggccaacatgaggatcacccatgtctgcagggcc

SEQ ID NO: 9 - RNA, 122 nt, synthetic construct
  source 1..122: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 120: note = 2-O-methyl ribose moiety and phosphorothioate
  misc_feature 121: note = 2-O-methyl ribose moiety and phosphorothioate
  misc_feature 122: note = 2-O-methyl ribose moiety and phosphorothioate
ccaacatgaggatcacccatgtctgcagggcctagcaagttaaaataaggctagtccgttatcaacttggccaacatgaggatcacccatgtctgcagggccaagtggcaccgagtcggtgc

SEQ ID NO: 10 - RNA, 34 nt, synthetic construct
  source 1..34: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 32: note = 2-O-methyl ribose moiety and phosphorothioate
  misc_feature 33: note = 2-O-methyl ribose moiety and phosphorothioate
  misc_feature 34: note = 2-O-methyl ribose moiety and phosphorothioate
ggtgttcaaagacgcttctggttttagagctagg

SEQ ID NO: 11 - RNA, 33 nt, synthetic construct
  source 1..33: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 31: note = 2-O-methyl ribose moiety and phosphorothioate
  misc_feature 32: note = 2-O-methyl ribose moiety and phosphorothioate
  misc_feature 33: note = 2-O-methyl ribose moiety and phosphorothioate
gtcaggaaagtccaacgccgttttagagctagg

SEQ ID NO: 12 - RNA, 33 nt, synthetic construct
  source 1..33: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 32: note = 2-O-methyl ribose moiety and phosphorothioate
  misc_feature 31: note = 2-O-methyl ribose moiety and phosphorothioate
  misc_feature 33: note = 2-O-methyl ribose moiety and phosphorothioate
taacaggaaggattctgcagttttagagctagg

SEQ ID NO: 13 - RNA, 34 nt, synthetic construct
  source 1..34: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 32: note = 2-O-methyl ribose moiety and phosphorothioate
  misc_feature 33: note = 2-O-methyl ribose moiety and phosphorothioate
  misc_feature 34: note = 2-O-methyl ribose moiety and phosphorothioate
actgtaaaagatgtaaagaggttttagagctagg

SEQ ID NO: 14 - RNA, 33 nt, synthetic construct
  source 1..33: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 31: note = 2-O-methyl ribose moiety and phosphorothioate
  misc_feature 32: note = 2-O-methyl ribose moiety and phosphorothioate
  misc_feature 33: note = 2-O-methyl ribose moiety and phosphorothioate
gggaggtcctgtccggctcgttttagagctagg

SEQ ID NO: 15 - RNA, 33 nt, synthetic construct
  source 1..33: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 31: note = 2-O-methyl ribose moiety and phosphorothioate
  misc_feature 32: note = 2-O-methyl ribose moiety and phosphorothioate
  misc_feature 33: note = 2-O-methyl ribose moiety and phosphorothioate
cgaccccgagcagcggaaggttttagagctagg

SEQ ID NO: 16 - RNA, 33 nt, synthetic construct
  source 1..33: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 31: note = 2-O-methyl ribose moiety and phosphorothioate
  misc_feature 32: note = 2-O-methyl ribose moiety and phosphorothioate
  misc_feature 33: note = 2-O-methyl ribose moiety and phosphorothioate
cgtcgggatcaactcgaacgttttagagctagg

SEQ ID NO: 17 - RNA, 94 nt, synthetic construct
  source 1..94: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 92: note = 2-O-methyl ribose moiety and phosphorothioate
  misc_feature 93: note = 2-O-methyl ribose moiety and phosphorothioate
  misc_feature 94: note = 2-O-methyl ribose moiety and phosphorothioate
agcatagcaagttgaaataaggctagtccgttatcaacttggccaacatgaggatcacccatgtctgcagggccaagtggcaccgagtcggtgc

SEQ ID NO: 18 - RNA, 65 nt, synthetic construct
  source 1..65: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
  misc_feature 65: note = 2-O-methyl ribose moiety and phosphorothioate
ggtgttcaaagacgcttctggtttcagagctatgctgggccacatgaggatcacccatgtggccc

SEQ ID NO: 19 - RNA, 64 nt, synthetic construct
  source 1..64: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 62: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
gtcaggaaagtccaacgccgtttcagagctatgctgggccacatgaggatcacccatgtggccc

SEQ ID NO: 20 - RNA, 64 nt, synthetic construct
  source 1..64: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 62: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
taacaggaaggattctgcagtttcagagctatgctgggccacatgaggatcacccatgtggccc

SEQ ID NO: 21 - RNA, 65 nt, synthetic construct
  source 1..65: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
  misc_feature 65: note = 2-O-methyl ribose moiety and phosphorothioate
actgtaaaagatgtaaagaggtttcagagctatgctgggccacatgaggatcacccatgtggccc

SEQ ID NO: 22 - RNA, 64 nt, synthetic construct
  source 1..64: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 62: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
gggaggtcctgtccggctcgtttcagagctatgctgggccacatgaggatcacccatgtggccc

SEQ ID NO: 23 - RNA, 64 nt, synthetic construct
  source 1..64: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 62: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
cgaccccgagcagcggaaggtttcagagctatgctgggccacatgaggatcacccatgtggccc

SEQ ID NO: 24 - RNA, 64 nt, synthetic construct
  source 1..64: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 62: note = 2-O-methyl ribose moiety and phosphorothioate
  misc_feature 63: note = 2-O-methyl ribose moiety and phosphorothioate
  misc_feature 64: note = 2-O-methyl ribose moiety and phosphorothioate
cgtcgggatcaactcgaacgtttcagagctatgctgggccacatgaggatcacccatgtggccc

SEQ ID NO: 25 - RNA, 126 nt, synthetic construct
  source 1..126: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 124: note = 2-O-methyl ribose moiety and phosphorothioate
  misc_feature 125: note = 2-O-methyl ribose moiety and phosphorothioate
  misc_feature 126: note = 2-O-methyl ribose moiety and phosphorothioate
gctgggccacatgaggatcacccatgtggcccagcatagcaagttgaaataaggctagtccgttatcaacttggccaacatgaggatcacccatgtctgcagggccaagtggcaccgagtcggtgc

SEQ ID NO: 26 - RNA, 33 nt, synthetic construct
  source 1..33: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 31: note = 2-O-methyl ribose moiety and phosphorothioate
  misc_feature 32: note = 2-O-methyl ribose moiety and phosphorothioate
  misc_feature 33: note = 2-O-methyl ribose moiety and phosphorothioate
ggtgttcaaagacgcttctggtttcagagctat

SEQ ID NO: 27 - RNA, 32 nt, synthetic construct
  source 1..32: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 30: note = 2-O-methyl ribose moiety and phosphorothioate
  misc_feature 31: note = 2-O-methyl ribose moiety and phosphorothioate
  misc_feature 32: note = 2-O-methyl ribose moiety and phosphorothioate
gtcaggaaagtccaacgccgtttcagagctat

SEQ ID NO: 28 - RNA, 31 nt, synthetic construct
  source 1..31: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 29: note = 2-O-methyl ribose moiety and phosphorothioate
  misc_feature 30: note = 2-O-methyl ribose moiety and phosphorothioate
  misc_feature 31: note = 2-O-methyl ribose moiety and phosphorothioate
taacaggaaggattctgcagtttcagactat

SEQ ID NO: 29 - RNA, 33 nt, synthetic construct
  source 1..33: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 31: note = 2-O-methyl ribose moiety and phosphorothioate
  misc_feature 32: note = 2-O-methyl ribose moiety and phosphorothioate
  misc_feature 33: note = 2-O-methyl ribose moiety and phosphorothioate
actgtaaaagatgtaaagaggtttcagagctat

SEQ ID NO: 30 - RNA, 32 nt, synthetic construct
  source 1..32: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 30: note = 2-O-methyl ribose moiety and phosphorothioate
  misc_feature 31: note = 2-O-methyl ribose moiety and phosphorothioate
  misc_feature 32: note = 2-O-methyl ribose moiety and phosphorothioate
gggaggtcctgtccggctcgtttcagagctat

SEQ ID NO: 31 - RNA, 32 nt, synthetic construct
  source 1..32: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 30: note = 2-O-methyl ribose moiety and phosphorothioate
  misc_feature 31: note = 2-O-methyl ribose moiety and phosphorothioate
  misc_feature 32: note = 2-O-methyl ribose moiety and phosphorothioate
cgaccccgagcagcggaaggtttcagagctat

SEQ ID NO: 32 - RNA, 32 nt, synthetic construct
  source 1..32: mol_type = other RNA
  misc_feature 1: note = 2-O-methyl ribose moiety and phosphorothioate
  misc_feature 2: note = 2-O-methyl ribose moiety and phosphorothioate
  misc_feature 3: note = 2-O-methyl ribose moiety and phosphorothioate
  misc_feature 30: note = 2-O-methyl ribose moiety and phosphorothioate
  misc_feature 31: note = 2-O-methyl ribose moiety and phosphorothioate
  misc_feature 32: note = 2-O-methyl ribose moiety and phosphorothioate
cgtcgggatcaactcgaacgtttcagagctat